(19) United States
(12) Patent Application Publication
(10) Pub. No.: US 2005/0058245 A1
(43) Pub. Date: Mar. 17, 2005

(54) Intensity-modulated radiation therapy with a multilayer multileaf collimator

(76) Inventor: Moshe Ein-Gal, Ramat Hasharon (IL)

Correspondence Address: Dekel Patent Ltd.;Beit HaRof'im, Room 27, 18 Menuha VeNahala Street, Rehovot, IL

(21) Appl. No.: 10/659,253

(22) Filed: Sep. 11, 2003

(51) Int. Cl. A61N 5/10

(52) U.S. Cl. 378/65

(57) ABSTRACT

An intensity modulated radiation therapy (IMRT) system including a radiation beam delivery device positionable in a plurality of spatial orientations, an IMRT control system adapted to modulate at least an intensity of a radiation beam emanating from the radiation beam delivery device depending on at least one of the spatial orientations of the radiation beam delivery device and in accordance with an IMRT intensity map, and a multilayer multileaf collimator placed in a path of the radiation beam emanating from the radiation beam delivery device, the multilayer multileaf collimator including a plurality of layers of radiation blocking leaves, the layers being at different positions along the path of the radiation beam generally traverse to the radiation beam.

### FIELD OF THE INVENTION

The present invention relates generally to systems and methods for intensity-modulated radiation therapy (IMRT), and particularly to IMRT performed with a multilayer multileaf collimator, such as but not limited to, in a non-segmented, dynamic technique.

### BACKGROUND OF THE INVENTION

A well-known family of radiation therapy devices generally includes a gantry that can be swiveled around a horizontal axis of rotation in the course of a therapeutic treatment. A linear accelerator may be located in the gantry for generating a high-energy radiation beam for therapy. During treatment, this radiation beam is trained on a zone of a patient lying in the isocenter of the gantry rotation.

The point of such therapy is to concentrate radiation on tumors or other target zones, but minimize radiation dosages applied to adjacent healthy tissue, especially certain parts of the body (e.g., the optic nerve) that are more sensitive to radiation. A radiation source directs radiation towards the target zone. By moving the radiation source along an arc over a period of time, the radiation is on the target during the entire movement along the arc. However, healthy tissue adjacent the tumor (such as between the tumor and source, and tissue past the tumor along the beam path) fortunately may receive radiation for only a small portion of the time, different sections of healthy tissue being in the radiation path at different places along the arc. Another approach is a step-and-shoot method, wherein radiation is emitted from a number of (stationary) orientations.

An important factor in such radiation treatment is maintaining the beam from the radiation source on the target zone. Precise positioning and shaping of the radiation source relative to the patient is thus required. The time of treatment affects the accuracy of the beam. A longer treatment time increases the chances that the patient or portion of the patient will move. Therefore, a shorter period of treatment is generally preferable because the chances of movement occurring are reduced.

To control the radiation emitted toward an object, a beam-shaping device, such as a plate arrangement or a collimator, is typically provided in the trajectory of the radiation beam between the radiation source and the object. A collimator is a beam-shaping device that may include multiple leaves, for example, a plurality of relatively thin plates or rods, typically arranged as opposing leaf pairs. The plates themselves are formed of a relatively dense and radiation impervious material and are generally independently positionable to delimit the radiation beam.

Multileaf collimators have multiple leaf or finger projections that may be moved individually into and out of the path of the radiation beam. The multiple leaves may be programmed to follow the spatial contour of the tumor as seen by the radiation beam as it passes through the tumor, or the “beam's eye view” (BEV) of the tumor during the rotation of the radiation beam source, which is mounted on a rotatable gantry of the linear accelerator. The multiple leaves of the multileaf collimator form an outline of the tumor shape as presented by the tumor volume in the direction of the path of travel of the radiation beam, and thus block the transmission of radiation to tissue disposed outside the tumor's spatial outline as presented to the radiation beam, dependent upon the beam's particular radial orientation with respect to the tumor volume.

Intensity modulated radiation therapy (IMRT) is a cancer treatment method that generally utilizes a multi-leaf collimator, and which delivers high doses of radiation to predefined targets while effectively sparing the surrounding tissues. Some examples of IMRT systems are described, for instance, in U.S. Pat. Nos. 6,052,435 and 6,449,336. IMRT has the capability of generating a dose distribution and of providing specific sparing of sensitive normal structures within complex treatment geometries. Unlike conformal treatment where the radiation from a given orientation is uniform, IMRT delivers modulated i.e., spatially varying radiation. Typically, the modulation takes the form of a matrix and the intensities are determined by an intensity map (IM) matrix. The intensity maps of the treatment beams may be optimized using an optimization algorithm, and each intensity map may be decomposed into a number of segments using a leaf-sequencing algorithm. The intensity maps, one per orientation, are derived as a solution to an optimization problem defined by the geometry of the target, the irradiation orientations and the physician's specifications. A typical IM matrix may have hundreds or thousands (or more) entries representing the intensities in small, equally-sized rectangular apertures.

IMRT can be implemented either in a dynamic approach or a segmented, step-and-shoot approach. In the dynamic approach, a sliding window for the radiation beam to pass through is formed by the multileaf collimator. This window is dynamically changed as the radiation arm or gantry sweeps around the patient. For example, one pair of the multileaf collimator leaves may move in the same direction at different speeds. The location and speed of each leaf-pair may be controlled (with some restrictions) in accordance with the particular intensity map for the spatial orientation of the radiation beam delivery system. This creates a sweeping opening for the radiation beam. Because the leaves travel at different speeds, the opening varies in size during the sweeping. Usually, elaborate speed control is needed for intensity modulated treatment. The speed control is needed for accurately defining the changing opening size.

In the step-and-shoot approach, a sequence of apertures is formed in accordance with a segmented IM. Sequences of multileaf-collimated, varying-intensity beams combine from each orientation to create a dose distribution in and around the target. Since the number of such radiation segments is practically not greater than 10 for each orientation, the optimized IM has to be approximated by a sum of segments, that is, uniform fields capable of being delivered using the multileaf collimator. The step-and-shoot approach obviates the need to control the speed of the leaves, but its performance is compromised due to segmentation of the IM.

### SUMMARY OF THE INVENTION

The present invention seeks to provide novel apparatus and methods for performing IMRT with a multilayer multileaf collimator, such as but not limited to, in a non-segmented, dynamic approach, as are described in detail hereinbelow.

There is thus provided in accordance with an embodiment of the invention an intensity modulated radiation therapy (IMRT) system including a radiation beam delivery device positionable in a plurality of spatial orientations, an IMRT control system adapted to modulate at least an intensity of a radiation beam emanating from the radiation beam delivery device depending on at least one of the spatial orientations of the radiation beam delivery device and in accordance with an IMRT intensity map, and a multilayer multileaf collimator placed in a path of the radiation beam emanating from the radiation beam delivery device, the multilayer multileaf collimator including a plurality of layers of radiation blocking leaves, the layers being at different positions along the path of the radiation beam generally traverse to the radiation beam.

In accordance with an embodiment of the invention the multilayer multileaf collimator includes a plurality of x-leaves of a first layer in a longitudinal direction, and a plurality of y-leaves of a second layer in a cross-over direction angled with respect to the longitudinal direction at an angle in a range of 0 to 90 degrees inclusive. Columns and rows of the IMRT intensity map (IM) correspond to widths of the y-leaves and x-leaves, respectively.

Further in accordance with an embodiment of the invention an IM cell is defined as the intersection of one of the columns and rows, the radiation beam emanating from the radiation beam delivery device passing through the IM cell, and radiation to the IM cell is selectively blocked by the y-leaves and x-leaves.

Still further in accordance with an embodiment of the invention the radiation beam delivery device is rotatable about a longitudinal axis by a motor and the leaves are movable by at least one actuator, and the IMRT control system is operative to control operation of the motor and the at least one actuator.

There is also provided in accordance with an embodiment of the invention a method for preparing a system to perform intensity modulated radiation therapy (IMRT), the method including providing a radiation beam delivery device positionable in a plurality of spatial orientations, and capable of delivering a radiation beam in accordance with an IMRT intensity map, and providing a multilayer multileaf collimator in a path of the radiation beam emanating from the radiation beam delivery device, the multilayer multileaf collimator including a plurality of layers of radiation blocking leaves, the layers being at different positions along the path of the radiation beam generally traverse to the radiation beam. An intensity modulated radiation beam may be delivered through an aperture defined by spacing between leaves of layers of the multilayer multileaf collimator.

### BRIEF DESCRIPTION OF THE DRAWINGS

The present invention will be understood and appreciated more fully from the following detailed description taken in conjunction with the drawings in which:

FIG. 1 is a simplified pictorial illustration of an IMRT system, constructed and operative in accordance with an embodiment of the present invention;

FIGS. 2A and 2B are simplified pictorial illustrations of a multilayer multileaf collimator used in the IMRT system of FIG. 1, constructed and operative in accordance with an embodiment of the present invention, in two different cycle positions during a treatment plan;

FIG. 3 is a simplified flow chart of a method for performing non-segmented IMRT, in accordance with an embodiment of the present invention; and

FIG. 4 is a simplified pictorial illustration of an arrangement of cells for performing IMRT, in accordance with an embodiment of the present invention.

### DETAILED DESCRIPTION OF THE PREFERRED EMBODIMENT

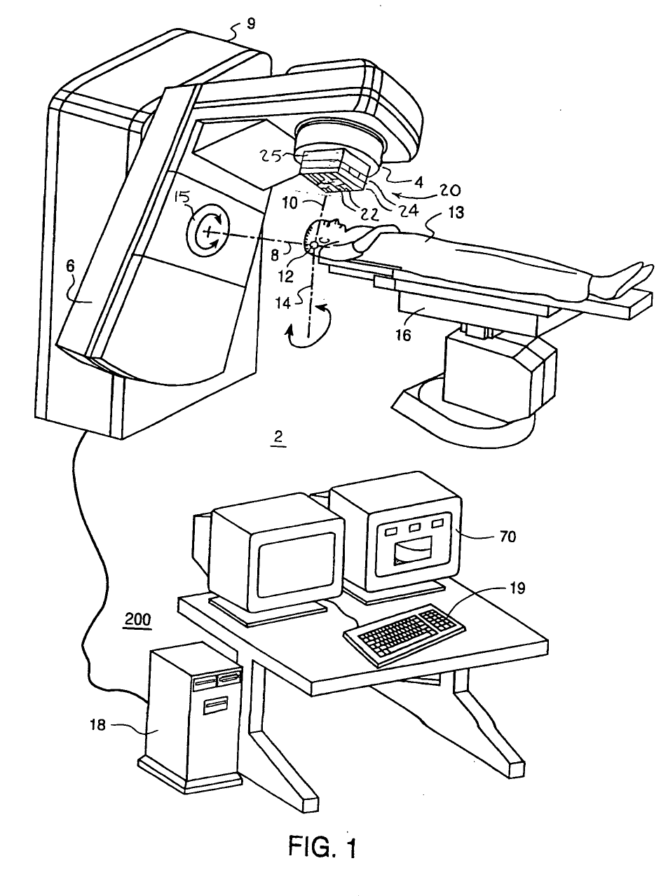

Reference is now made to FIG. 1, which illustrates an IMRT system 2, constructed and operative in accordance with an embodiment of the present invention.

IMRT system 2 may comprise a radiation beam delivery device 6, such as a gantry of an irradiation device 9 (e.g., a LINAC [linear accelerator] system). Radiation beam delivery device 6 is positionable in a plurality of spatial orientations. For example, radiation beam delivery device 6 may be rotated about a longitudinal axis 8, such as by means of a motor 15 and the like. A treatment head 4 may be fastened to a portion of radiation beam delivery device 6, containing a source of radiation for producing a beam of radiation 10, such as but not limited to, electron, photon or any other radiation used in therapy.

During the treatment, the radiation beam 10 is trained on a target 12 of an object 13, for example, a patient who is to be treated. The longitudinal axis 8, a rotational axis 14 of a treatment table 16, and the beam axis of the beam 10 intersect at a point called the isocenter. The patient is positioned so that the isocenter lies in the target 12.

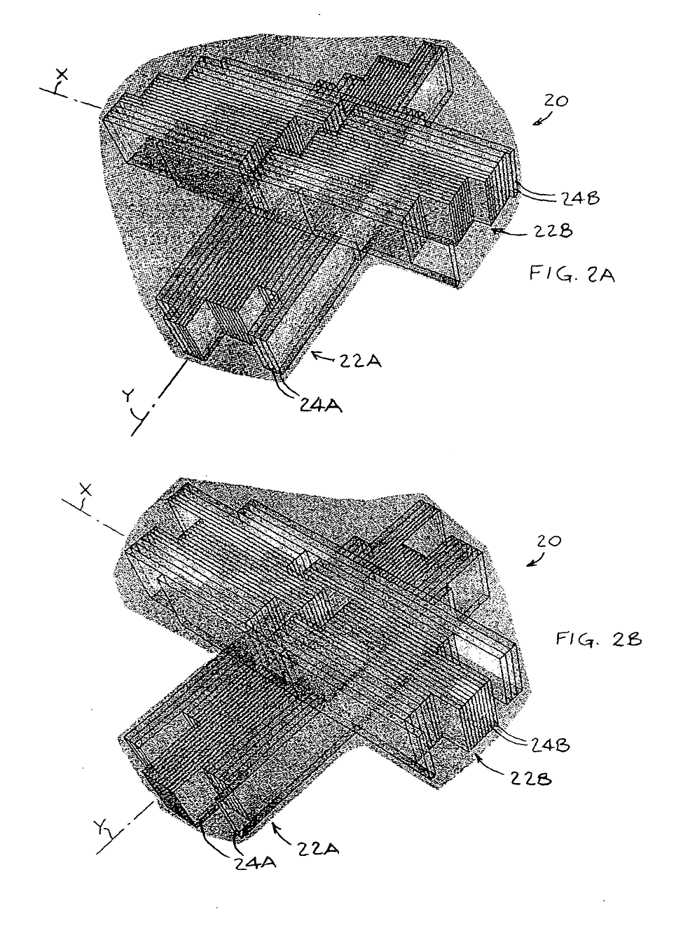

A multilayer multileaf collimator 20 may be placed in a path of the radiation beam 10 emanating from radiation beam delivery device 6. For example, multilayer multileaf collimator 20 may be secured to treatment head 4. Multilayer multileaf collimator 20 may comprise a plurality of layers 22 of radiation blocking leaves 24 (described more in detail hereinbelow with reference to FIG. 2). It is emphasized that the term “leaves” is not limited to leaf-like structure, rather the term “leaves” encompasses any kind of radiation blocking structure, such as but not limited to, rods, plates, and the like, of any size and shape. The layers 22 may be at different positions along the path of radiation beam 10, generally traverse to radiation beam 10. The leaves 24 are movable such that the distribution of radiation over the field need not be uniform (one region may be given a higher dose than another). Furthermore, radiation beam delivery device 6 may be rotated so as to allow different beam angles and radiation distributions without having to move the patient 13.

An IMRT control system 200 may be provided (in the same room or remotely located) to modulate at least an intensity of the radiation beam 10, depending on at least one of the spatial orientations of radiation beam delivery device 6 and in accordance with an IMRT intensity map. IMRT control system 200 may include output devices 70, such as but not limited to, one or more visual display units or monitors, and input devices 19, such as but not limited to, a keyboard or mouse. Data may also be input through data carriers, such as data storage devices or a verification and recording or automatic setup system. IMRT control system 200 may also control, without limitation, rotational speed of radiation beam delivery device 6 about longitudinal axis 8, by controlling operation of motor 5, for example.

IMRT control system 200 may typically be operated by a therapist (not shown) who administers actual delivery of radiation treatment as prescribed by an oncologist (not shown) by using input device 19. The therapist enters into IMRT control system 200 the data that defines the radiation dose to be delivered to the patient, for example, according to the prescription of the oncologist. The information may alternatively or additionally be input via another input device, such a data storage device. Various data may be displayed before and during the treatment on the screen of the output device 70.

Reference is now made to FIGS. 2A and 2B, which illustrate an example of multilayer multileaf collimator 20. Multilayer multileaf collimator 20 may comprise a cross-multi-micro-leaves collimator (CMMLC). For example, leaves 24A of a first layer 22A may be in the longitudinal direction (Y), and leaves 24B of a second layer 22B may be in the cross-over direction (X). The first and second layers 22A and 22B are arranged one above another in an overlapping manner in the beam direction. One or more actuators 25 (e.g., a step motor, linear encoder and the like), seen in FIG. 1, may be used to move the leaves in accordance with a treatment plan. One actuator 25 may be dedicated to moving one or more leaves. IMRT control system 200 may control operation of actuator 25.

The illustrated CMMLC is a two-layer MLC where the leaves in one layer (x-leaves) are generally perpendicular to the ones in the other (y-leaves). However, it is emphasized that the present invention is not limited to this construction, and may be carried out with any number of layers and leaves and at any angle therebetween.

In one embodiment, the geometry of the intensity map is such that the columns and the rows correspond to the actual width of the y-leaves and x-leaves, respectively. For example, if the CMMLC has 24 pairs of x-leaves and 24 pairs of y-leaves (not necessarily with equal width), then the dimension of the intensity map may be 24×24, 12×12, 8×8 etc., depending on whether a row or column corresponds to one, two or three adjacent leaves, respectively. This is referred to as the CMMLC/IM geometry.

Defining an IM cell as the intersection of a row and a column, this IM arrangement allows blocking the radiation to a cell by an x-leaf or a y-leaf or both. This enables performing non-segmented IMRT, that is, radiation delivery according to the optimized intensity maps without segmentation. (However, it is noted that the invention is not limited to non-segmented use, and may be used for the segmented approach as well.)

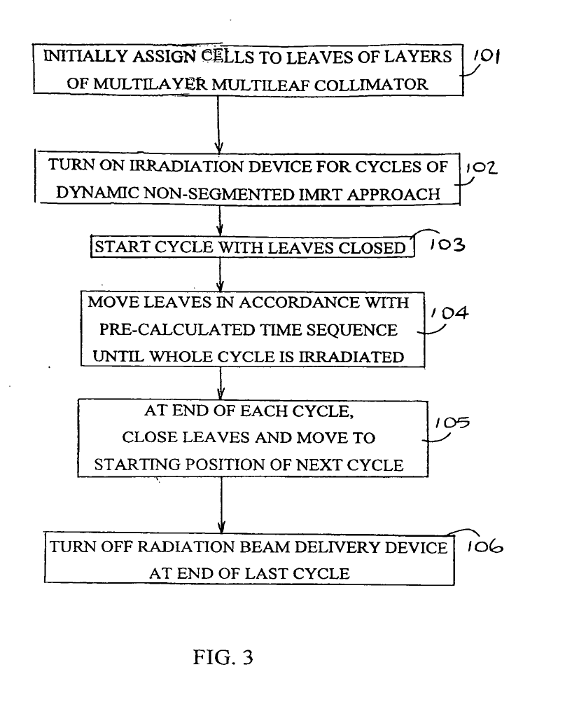

An example of a method for performing non-segmented IMT is now described with reference to FIG. 3. It is understood that this is just one example of carrying out the invention and the invention is not limited to this example.

The following definitions are useful in understanding this example:

A cell: an intersection of a column, a row and the target (PTV).

A 2cell: a sub-matrix of 2×2 cells.

A 4cell: a sub-matrix of 2×2 2cells.

A cycle: N 4cells with different rows and columns (which may be exposed simultaneously), wherein N is the number of cycles in the intensity map. N may determine the overall irradiation time, since N cycles may be irradiated sequentially.

It may be seen from FIGS. 2A and 2B that the two layers 22A and 22B of perpendicular leaves 24A and 24B have a slightly different ratio of distance-to-source/distance-to-isoplane (i.e., the plane of the isocenter) and, therefore, different projections of the same leaf width.

Initially, the cells may be assigned to leaves 24 of the layers 22 (step 101, FIG. 3). For example, each 4cell may be assigned 16 leaves, 4 leaves per each 2cell, N (all) leaves per cycle. The irradiation device 9 (e.g., LINAC) may be turned on for all of the cycles of the dynamic non-segmented IMRT approach (step 102, FIG. 3). The intensity map may be implemented (exposed) cycle after cycle once irradiation device 9 is turned on. For example, a cycle may start with all leaves closed (step 103, FIG. 3). Afterwards, the leaves may be moved in accordance with a pre-calculated time sequence (step 104, FIG. 3) until the whole cycle is irradiated. For example, as seen in FIG. 2A, the leaves of both layers have been moved to define an aperture 26 to implement one cycle of the IM. As seen in FIG. 2B, the leaves of both layers have been moved to define a different aperture 28 to implement another cycle of the IM.

At the end of each cycle, the leaves may close again and move to the starting position of the next cycle (step 105, FIG. 3). The LINAC may be turned off at the end of the last cycle (step 106, FIG. 3). During the cycles, radiation beam delivery device 6 may rotate about longitudinal axis 8 at any constant or varying velocity.

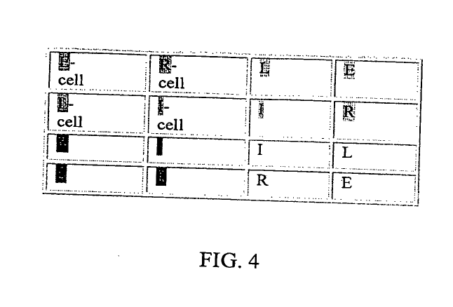

Reference is now made to FIG. 4, which illustrates an arrangement of the cells for performing IMRT in accordance with an embodiment.

The following is an example of an irradiation sequence that may be employed with the arrangement of FIG. 4. It is sufficient to derive the irradiation sequence algorithm for one 2cell due to homomorphism of all of the 2cells.

The cells in a 2cell are termed I (internal), L (left), E (external) and R (right) in a clockwise fashion where I is the cell closest to the 4cell center. The cells in a 2cell are ordered (1 to 4) according to corresponding IM values where 1 signifies the least amount. For example, (1234=) LERI signifies that the L-cell has the lowest IM value in the 2cell and the I-cell has the highest. There are 24 possible arrangements and each one is associated with an algorithm that takes into account the actual IM values. The output of such a 2cell algorithm is a time sequence for the position of the leaves corresponding to the IM values of the four cells comprising the 2cell. Since all 2cells may be irradiated simultaneously, the time-sequences of all 2cells in a cycle may concatenate into a cycle time sequence.

Prior to 2cell exposure, the four 2cell leaves may be positioned at the 2cell boundaries adjacent to other 2cells in a 4cell (2cell completely closed). At the start, all 4 2cell leaves may move to the 2cell boundary (open completely), allowing all four cells to be exposed. The leaves then close or close-and-open-and-close each cell according to a pre-calculated timing sequence until the 2cell irradiation is complete and the leaves are back at the 2cell closed position. (Again, it is noted that the invention is not limited to this example, and other arrangements may be used as well.)

Various parameters may affect calculation of the time sequence for a given IM, such as but not limited to, resolution, LINAC exposure rate, leaves speed, cell position, output factor, positioning accuracy, penumbra, leaves attenuation, and/or inter-leaves leakage.

The resolution may be, for example, high, low, mixed, or modified mixed. In the above-described example, the CMMLC has 4 banks of 24 leaves each. A high resolution of 24×24 for the IM (N=6) is thus available. In such a case, a cell size may be a few mm, and a cycle may have six 4cells, wherein the maximum number of cycles intersecting the target is six.

Another possibility may consider adjacent pairs of leaves as leaves. In such a case, the intensity map is 12×12 (N=3), and a cell size is close to one cm. There would be three 4cells in a cycle, and at most 3 cycles would be used to complete the irradiation, with lower resolution. It is noted that each 4cell may be of different resolution, as long as all 4cells in a cycle do not share a row or a column. This may enable a mixed resolution procedure, which is in between the high and the low ones, relative to both resolution and irradiation time. It enables treating the high contrast areas of the IM with high resolution and saving irradiation time by treating the low contrast areas of the IM with low resolution. The modified mixed approach amounts to starting with a 24×24 IM, combining cells (averaging) to form a 12×12 IM, and then manipulating the four leaves of the cell (two leaves) according to the 24×24 IM.

The LINAC exposure rate converts the required monitor units (MU) per cell as given by the IM into an exposure time, and thus determines the timing associated with the opening and closing of cells. In general, a lower rate is associated with increased accuracy but a longer treatment time. Requirements concerning accuracy, treatment time and LINAC limitations determine the desired exposure rate.

The leaves speed may be initially assumed constant, wherein the leaves motion causes the cell to have a linearly increased irradiation in one or two directions. Knowledge of this irradiation pattern enables a modification of the IM such that the optimization process takes into account the beam shape in each cell. The total amount of irradiation during motion depends on leaves speed and on cell size. In practice, leaves speed may slightly vary from one leaf to the next, from time to time, with LINAC rotation and with leaf motion direction. Cell size and leaves speed may determine opening or closing motion time. Irradiation during motion may be taken into account. The cells may be slightly expanded (by a margin) as a strategy for reducing penumbra effects.

It is noted that corrections may be made for cell position.

It may be assumed that the output factor (OF) increases with cell size due to the increased contribution of the distributed source to the cell. The difference between the output factor of a 1×1 cm cell and a 10×10 cm cell (when the leaves stay at 10×10 cm) is about 10%, for example. In addition, phantom scatter may also increase with cell size (variation might be about 5%, for example). Since the IM is calculated for a completely open field, the “output factor” due to scatter and distributed source may be calculated using an increasing (measured) function of cell size.

Errors in leaves positioning may cause increased or reduced irradiation at cells boundary. When the expected magnitude of the error is known, margins to each cell may be applied either for increased or reduced irradiation.

Penumbra (lateral scattering) may affect the exposure distribution in the irradiated object and reduce the exposure difference (contrast) between adjacent cells compared to the associated irradiation contrast. Ideally, the optimization process of the IM takes penumbra into account. Otherwise, other techniques may be used, such as but not limited to, enlarging cell size (leading to overlapping cells) to accommodate for loss of exposure at the cell boundary.

The cross-MMLC allows the irradiation of each cell to be blocked by one or two leaves. Two-leaves attenuation is expected to be about 99% while one-leaf attenuation is about 90%. Since irradiation is always present (in the dynamic approach), the contribution of the attenuated irradiation may affect the irradiation time sequence.

Inter-leaves leakage may contribute irradiation to cells boundaries, and may be taken into account.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. An intensity modulated radiation therapy (IMRT) system comprising:
a radiation beam delivery device positionable in a plurality of spatial orientations;
an IMRT control system adapted to modulate at least an intensity of a radiation beam emanating from said radiation beam delivery device depending on at least one of the spatial orientations of said radiation beam delivery device and in accordance with an IMRT intensity map; and
a multilayer multileaf collimator placed in a path of the radiation beam emanating from said radiation beam delivery device, said multilayer multileaf collimator comprising a plurality of layers of radiation blocking leaves, said layers being at different positions along said path of the radiation beam generally traverse to the radiation beam.

2. The system according to claim 1, wherein said multilayer multileaf collimator comprises a plurality of x-leaves of a first layer in a longitudinal direction, and a plurality of y-leaves of a second layer in a cross-over direction angled with respect to said longitudinal direction at an angle in a range of 0 to 90 degrees inclusive.

3. The system according to claim 2, wherein columns and rows of said IMRT intensity map (IM) correspond to widths of said y-leaves and x-leaves, respectively.

4. The system according to claim 3, wherein an IM cell is defined as the intersection of one of said columns and rows, the radiation beam emanating from said radiation beam delivery device passing through said IM cell, and radiation to the IM cell is selectively blocked by said y-leaves and x-leaves.

5. The system according to claim 1, wherein said radiation beam delivery device is rotatable about a longitudinal axis by a motor and said leaves are movable by at least one actuator, and said IMRT control system is operative to control operation of said motor and said at least one actuator.

6. A method for preparing a system to perform intensity modulated radiation therapy (IMRT), the method comprising:
providing a radiation beam delivery device positionable in a plurality of spatial orientations, and capable of delivering a radiation beam in accordance with an IMRT intensity map; and
providing a multilayer multileaf collimator in a path of the radiation beam emanating from said radiation beam delivery device, said multilayer multileaf collimator comprising a plurality of layers of radiation blocking leaves, said layers being at different positions along said path of the radiation beam generally traverse to the radiation beam.

7. The method according to claim 6, further comprising delivering an intensity modulated radiation beam through an aperture defined by spacing between leaves of layers of said multilayer multileaf collimator.
